(19) United States
(12) Patent Application Publication
(10) Pub. No.: US 2024/0197574 A1
(43) Pub. Date: Jun. 20, 2024

(54) LARGE BORE NASOGASTRIC TUBE HOLDER

(71) Applicant: Dale Medical Products, Inc., Franklin, MA (US)

(72) Inventors: Ronald D. Russo, Naples, FL (US); Joseph A. Zilaro, Sturbridge, MA (US)

(21) Appl. No.: 18/396,193

(22) Filed: Dec. 26, 2023

Related U.S. Application Data
(60) Provisional application No. 63/381,948, filed on Nov. 2, 2022.

(51) Int. Cl. A61J 15/00; A61M 25/02

(52) U.S. Cl. CPC A61J 15/0061; A61J 15/0003; A61M 2025/0226; A61M 2025/0253

(57) ABSTRACT

A large bore patient's nasogastric tube holder is disclosed. The holder is fabricated from a heavy duty elasticized fabric having a high tack synthetic rubber adhesive backing covered with Kraft release liners. The holder comprises an adhesive backed upper nose base portion, a central bridge portion, and a singular lower tube securement tab. The central bridge portion also having a non-absorbent rear surface area including a cushioned structurally supportive pad preventing nasal acquired pressure injury (NAPI). The bridge portion extending into a sideways connector portion which positions the singular lower tube securement tab alongside and parallel to a tube exiting a patient's nare. The lower tube securement tab providing a secure spiral wrap around the tube to keep the tube centered within a patient's nare to prevent twisting and torquing of the tube and nasal irritation.

### CROSS REFERENCE TO RELATED APPLICATION(S)

This application claims the benefit of U.S. Provisional Application No. 63/381,948, entitled “LARGE BORE NASOGASTRIC TUBE HOLDER” and filed on Nov. 2, 2022, the disclosure of which is expressly incorporated by reference herein in its entirety.

### BACKGROUND OF THE INVENTION

Patients requiring gastric decompression and draining of stomach contents are often intubated with a nasogastric tube. The tubes are often a double lumen stomach tube termed a Salem Sump™ tube or a single lumen stomach tube termed a Levin tube. These tubes are usually attached to low level continuous suction or intermittent syringe suction to aspirate both air and liquid stomach contents. These gastric decompression tubes are referred to as large bore tubes since they range in size from 10 French up to 20 French with large bore flow paths.

They are typically fabricated of semi-rigid PVC plastic. Due to their stiffness, they have a tendency to twist and torque and often are difficult to control, manage, and keep in centered alignment within a patient's nare which causes nasal trauma and irritation. Further, these large bore plastic tubes are difficult to secure in place and can become dislodged during routine patient care.

Further, during routine patient care the tube is often pulled on or tension is applied to connect the tube to suction or an I.V. pole for administration of enteral formula. This tension produces internal nares pressure causing nasal acquired pressure injury (NAPI).

By comparison, small bore enteral feeing nasogastric tubes are typically made from flexible polyurethane and range in smaller sizes typically from 5 French up to 8 French in size. Most often clinicians use hand made and hand cut hospital tape to secure the large bore tubes, but do utilize commercial tube holders which are easier to manage and secure for the small bore enteral feeding tubes. As such, there really is a need to create a large bore nasogastric tube holder specifically created and designed to address the need for securement of the large bore difficult to manage nasogastric stomach tubes.

Therefore, there is a need for a device which is targeted at securing the large bore nasogastric tubes, but can also be utilized to secure small bore enteral feeding tubes as well. Towards this end, the present invention of a large bore nasogastric tube holder has been conceived and reduced to practice to help prevent nasal acquired pressure injury (NAPI).

### SUMMARY OF THE INVENTION

In accordance with the present invention, a unique large bore nasogastric tube holder is invented and disclosed for use on a patient. The holder is fabricated from an elasticized fabric having an aggressive yet skin friendly synthetic rubber adhesive backing covered with a Kraft release liner. The holder is configured with an upper nose base, a central bridge portion, a sideways connector portion extending from the central bridge portion which orients a lower singular tube securement tab alongside the intubated tube.

The holder is manufactured in a linear fashion using automatic tape converting equipment capable of in-line overlaying different materials and then finally die cutting the finished assembly in one piece. The nose base has an upper adhesive backed portion for adhesive application to the patient's nose. The nose base portion also has a lower portion transitioning to a central bridge portion which is further structured to include a cushioned structurally supportive pad. The pad has a non-adherent and non-absorbent rear surface area preventing adhesion of the central bridge portion to a patient's nare. Extending from the pad is a sideways connector portion angled or bent at a 45 degree angle which extends into the singular tube securement tab.

The sideways connector portion permits orientation and alignment of the singular adhesive backed tube securement tab alongside and parallel to the tube. The singular tube securement tab provides a spiral adhesive backed securement of the nasogastric tube.

Additional attributes of the present invention will become more evident from a review of the accompanying drawings and detailed description.

### BRIEF DESCRIPTION OF THE DRAWINGS

FIG. 1 is a frontal view of the holder.

FIG. 2 is a side layered fabricated view of the holder depicting the application of the structurally supportive pad.

FIG. 3 is a rear view of the holder depicting the pad on the central bridge portion.

FIG. 4 is a frontal view of the holder applied to a patient's nose and the sideways connector portion aligning the lower securement tab alongside and parallel to the tube.

FIG. 5 is a frontal view of the holder depicted from FIG. 4 wherein the lower securement tab is spiral wrapped around the tube.

FIG. 6 is a frontal view of a typical commercial holder wherein pulling action on the tube creates internal nasal acquired pressure injury (NAPI).

FIG. 7 is a frontal view of the present invention holder wherein the structurally supportive central bridge pad helps to prevent internal nasal acquired pressure injury when pulling action is applied to the tube.

FIG. 8 is the application and removal instructions of the present invention.

FIG. 9 is a frontal view of the present invention depicting the nose base having a full curvilinear top configuration and the rear surface structurally supportive pad being used as a non-adherent finger grip area to hold the device steady in position to aid in spiral wrapping the singular securement tab around the tube.

FIG. 10 is a rear view of the present invention depicting how the rear surface structurally supportive non-adherent pad separates the upper nose base from the lower singular tube securement tab providing easier application and removal of both the base and tab.

### DETAILED DESCRIPTION OF THE INVENTION:

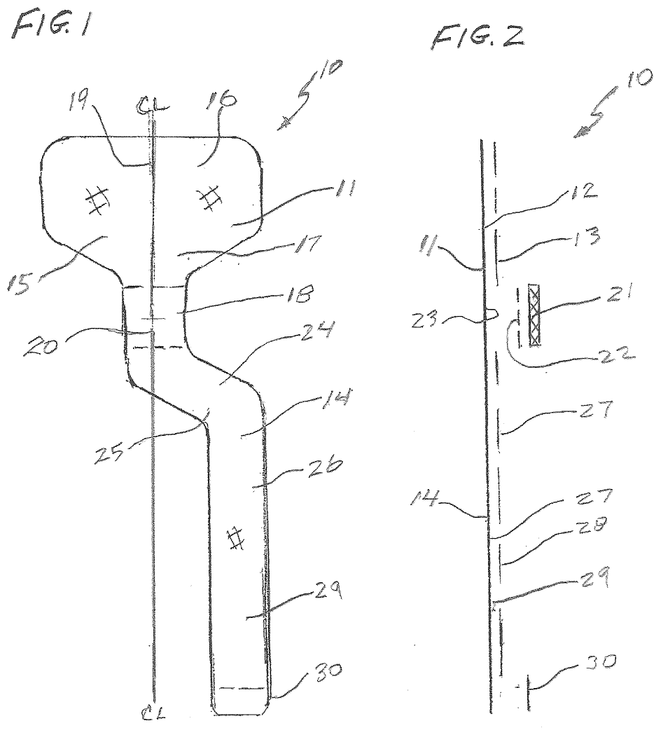

FIG. 1 is a frontal view of large bore nasogastric tube holder 10 and FIG. 2 illustrates the layered view of holder 10. The entire frontal material 11 of holder 10 is constructed from a woven tan fabric matrix of cotton and rayon with a non-latex rubber based adhesive backing 12 supplied on a Kraft paper release liner 13. It should be noted that the entire facing 14 of holder 10 is constructed of tan fabric material 11.

Holder 10 is structured with nose base 15 having an upper adhesive backed portion 16 and a lower portion 17 which transitions into a central bridge portion 18. Bridge portion 18 is centered and centrally aligned with nose base center line 19. The central bridge portion 18 is important in that it assists in visually positioning and aligning the nose base 15 to the side of the nose where the nasogastric tube exits the nose and keeping the tube centrally aligned with center line 20 on nose base lower portion 17.

Another important aspect of bridge portion 18 is that it is constructed of a 1/32 inch thick closed cell non-absorbent polyethylene foam stiffening pad material 21 which is laminated to fabric material 11 using adhesive 22 on material backside 23. Bridge 18 is therefore assembled onto the backside 23 of holder 10. As such, pad 21 now provides a structurally supportive cushioned non-adherent rear surface area. While pad 21 is preferably cushioned, other non-cushioned non-adherent materials such as 3 mil polyester film or synthetic tape may also provide a structurally supportive non-adherent rear pad surface area.

The polyethylene foam stiffening non-absorbent material pad 21 further acts as a structural bridge support and keeps the nose base portions 16 and 17 in place on the nose and prevents twisting and torquing of the nasogastric tube within a patient's nare which can cause nasal acquired pressure injury (NAPI). As such, the non-adherent rear backside surface area 23 on central bridge portion 18 includes and provides a cushioned structurally supportive tube pad 21 preventing adhesion of the central bridge portion 18 to a patient's nare as well as a structurally supportive pad. The central bridge portion 18 having a non-absorbent cushioned rear surface area 23 now includes a structurally supportive tube pad 21 to help prevent NAPI and to keep the tube centrally aligned within a patient's nose nare when the tube is subjected to external lateral pulling action.

Non-absorbent foam material pad 21 also prevents absorption of nasal secretions onto the bridge portion 18. Bridge portion 18 further transitions into a sideways connector portion 24 structured in a downward and bent position in relationship to the center line 20 on nose base lower portion 17. Sideways connector portion 24 flows into a 45 degree angle 25 and continues on to permit connection to a singular tube securement tab 26. Both sideways connector portion 24 and tab 26 are adhesive backed 27 with Kraft paper release liner 28. Securement tab 26 has an elongated adhesive backed extension 29 terminating in non-adherent plastic 2 mil polyester end tip 30 laminated onto the end of extension 29.

Non-adherent end tip 30 will aid the clinician in manually grasping the end of extension 29 during set up, application, and removal of the holder 10 from the patient.

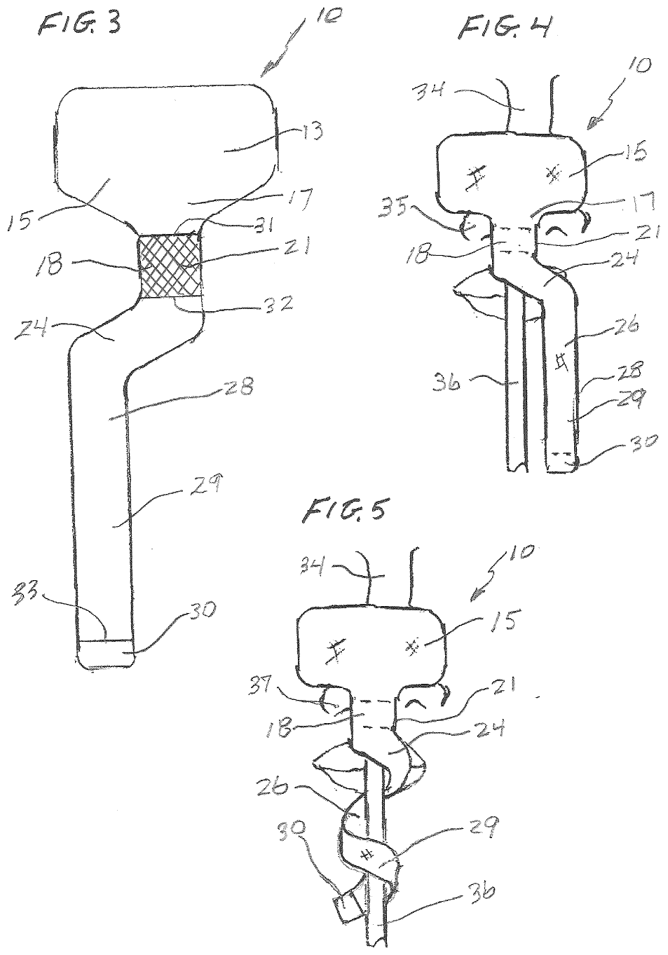

FIG. 3 is a rear view of holder 10 wherein lower nose base portion 17 has a back paper release liner 13 incorporating a top lift edge 31 and a lower lift edge 32 which abuts the top and lower edges of center non-adherent foam bridge portion 18 incorporating structurally supportive pad 21.

Lift edges 31 and 32 provide convenient manual removal lift edges of rear side Kraft paper release liners 13 and 28 Likewise, lift edge 33 on lower release liner 28 which abuts non-adherent tip 30 also creates a convenient manual removal lift edge for lower release liner 28.

FIG. 4 is a frontal view of the holder 10 applied to a patient's nose 34 wherein the center bridge portion 18 assists in positioning and aligning nose base 15 to the side 35 where the nasogastric tube 36 exits the nose 34. In essence, center line alignment and positioning of the center bridge portion 18 with respect to nasogastric tube 36 will automatically correctly position both the off-set bridge portion 24 and the singular tube securement tab 26 alongside and in a parallel fashion with the nasogastric tube 36.

Sideways connector portion 24 is structured in a downward and bent position from the nose base lower portion 17. It is bent at an approximate 45 degree angle from the center line 20 as shown in FIG. 1. That positioning and angled arrangement sets up a proper spiral adhesive securement orientation for both the off-set bridge portion 24 and singular tube securement tab 26 as depicted in spiral wrap securement arrangement in FIG. 5.

FIG. 5 clearly depicts the spiral wrap securement arrangement and method provided by the unique structure of the present invention holder 10 and also clearly depicts the rear surfaced cushioned non-adherent pad 21 preventing adhesion of the central bridge portion 18 to the patient's nare.

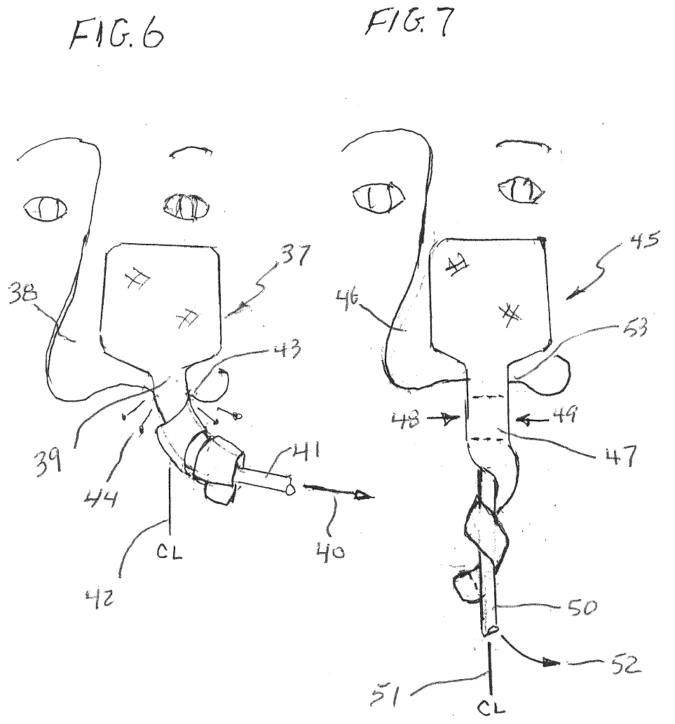

FIG. 6 is a frontal view of a typical commercially available prior art naso-gastric tube holder 37 adhesively applied to a patient's nose 38. As can be seen, since there is no structurally supportive bridge portion in area 39, external pulling action 40 on tube 41 causes the tube 41 to move off the center line 42 within the nare. The tube 40 now presses up against the internal nare 43 to create nasal acquired pressure injury 44.

By comparison, from FIG. 7 there is illustrated the holder of the present invention 45 adhesive applied to a patient's nose 46 in a similar manner as depicted in FIG. 6 wherein structurally supportive central bridge portion 47 acts in a compressive supportive structural manner 48 and 49 to help keep tube 50 centrally aligned with tube center line 51.

As such, external lateral pulling action 52 on tube 50 has little effect on twisting tube 52 within internal nare 53. Tube 50 thus is maintained within a central location within nare 53 and nasal acquired pressure injury is significantly reduced and prevented.

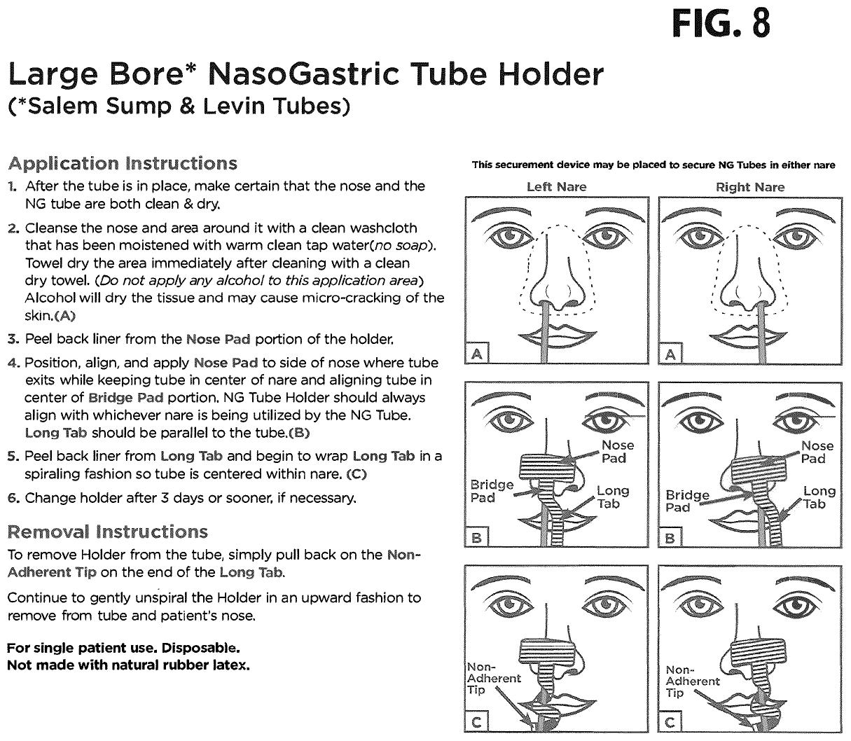

FIG. 8 further describes the set-up, application and removal instructions to the user. The instructions and accompanying figures clearly show and describe how the holder can be set up in either the left or right nare.

Further, As can be seen from both FIGS. 5 and 7, both the long singular tube securement tab 26 and elongated extension 29 firmly secure and align the tube 36 in the center of nare 37. Both tab 26 and extension 29 are combined for a length of at least 3 to 4 inches which provides a singular spiral wrap engagement with tube 36.

That engagement keeps the tube 36 centered within the nare 37 and prevents the tube 36 from twisting and torquing which causes nasal trauma and irritation and nasal acquired pressure injury (NAPI).

As noted in FIG. 8, the holder is for single patient use, disposable, and is not made with natural rubber.

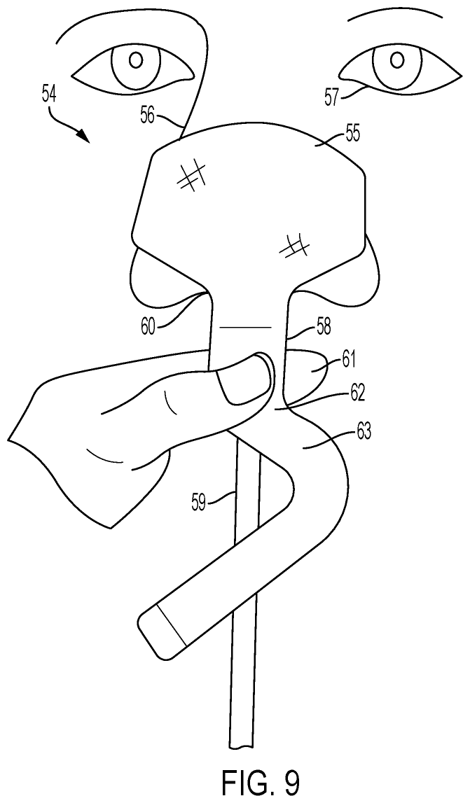

FIG. 9 clearly depicts the holder of the present invention 54 constructed with a full curvilinear top portion 55 around the bridge of the nose 56 and prevents the top portion 55 from contacting the eye area 57 on smaller faced patients.

Further, the non-adherent cushioned rear surface area pad 58 acts a s convenient finger grip area to hold and align the nasogastric tube 59 steady in place to aid in aligning the tube centrally within the patient's nare 60.

The pad 50 further permits manual finger grasping 61 of the tube 59 and bridge portion 62 to orient sideways connector portion 63 before beginning spiral wrapping action of the bridge portion 62 around the tube 59.

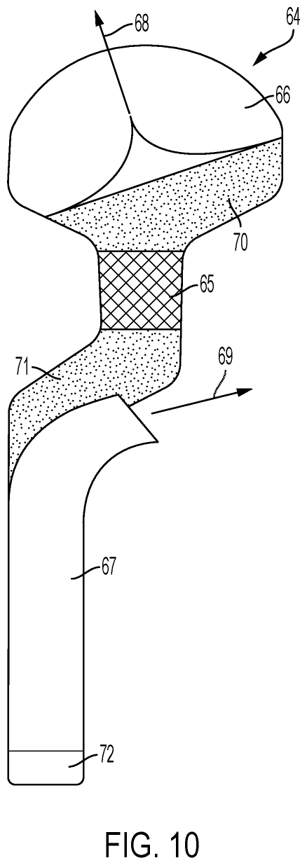

FIG. 10 is a rear view of the present invention holder 64 similar to the rear view of the holder in FIG. 3. As can be seen from FIG. 10, the non-absorbent rear surface area structurally supportive cushioned pad 65 isolates and functionally separates upper nose base 66 from lower singular tube securement tab 67. This separation provides for convenient and easier finger removal of the upper base liner 68 and lower tab liner 69 especially when using gloved fingers. Further, this separation provides individual exposure of upper nose base adhesive 70 and lower tab adhesive 71 without interference between the upper nose base 66 and lower tube securement tab 67 portions thereby making application and removal of both the nose base 66 and lower tube securement tab 67 less cumbersome.

This is especially important when un-wrapping lower tube securement tab 67 using mylar tip 22. Without the separation between upper base 66 and lower tab 67 provided by structural pad 65, lower tab 67 would be pulling tearing off upper nose base from the patient's nose.

As such the lower tab 67 can be un-wrapped from the tube first and the upper nose base 66 un-wrapped from the tube second, thereby producing less pain and injury to delicate nose skin tissue. The pad 65 functionally separates said upper nose base 66 from lower tube securement tab 67 such that both can function independently of each other.

The holder of the present invention provides a unique device for securement and management of large bore nasogastric tubes in the clinical situation.

It is contemplated and anticipated that different materials such as different tape, adhesives, or liners can easily be substituted for those shown and described without departing form the unique design, construction and clinical benefits of the underlying invention.

What is claimed is:

1. A holder for securement of a nasogastric tube exiting a patient's nose, said holder comprising;
a nose base, a central bridge portion, and a tube securement tab, said nose base having an upper adhesive backed portion for adhesive attachment of the upper portion to a patient's nose, and a lower portion transitioning into said central bridge portion, said central bridge portion having a non-absorbent rear surface area including a structurally supportive tube pad to help keep said tube centrally aligned within a patient's nose nare to help prevent nasal acquired pressure injury (NAPI) when said tube is subjected to external lateral pulling action.

2. The holder of claim 1 wherein the structurally supportive tube pad is cushioned.

3. The holder of claim 1 wherein said central bridge portion includes a sideways connector portion permitting connection to said tube securement tab.

4. The holder of claim 3 wherein the sideways connector portion permits the tube securement tab to be positioned alongside and parallel to the nasogastric tube exiting a patient's nose.

5. The holder of claim 1 wherein the tube securement tab terminates in a non-adherent end tip.

6. A holder for securement of a nasogastric tube exiting a patient's nose, said holder comprising;
A nose base, a central bridge portion, and a singular tube securement tab, said nose base having an upper adhesive backed portion for adhesive attachment of the upper portion to a patient's nose, and a lower portion transitioning into said central bridge portion, aid central bridge portion having a non-absorbent rear surface area including a structurally supportive tube pad, said central bridge portion further extending in a sideways connector portion, said sideways connector portion permitting connection with said singular tube securement tab, wherein said sideways connector portion further permits orientation and alignment of said singular tube securement tab alongside said tube exiting a patient's nare, said singular tube securement tab having an elongated adhesive backed extension, said extension aligned alongside and in a parallel fashion with said tube exiting a patient's nare as a result of said positioning, said singular tube securement tab extension providing an adhesive backed securement with said nasogastric tube exiting a patient's nare.

7. The holder of claim 6 wherein the non-absorbent rear surface area structurally supportive tube pad is cushioned.

8. The holder of claim 6 wherein the nose base has a full curvilinear top portion which permits better contouring of the top portion of the nose base around the bridge of the nose.

9. The holder of claim 6 wherein the non-absorbent rear surface area structurally supportive tube pad acts as a convenient finger grip area to hold and align the nasogastric tube steady in place to aid in aligning the tube centrally within the patient's nare.

10. A holder for securement of a nasogastric tube exiting a patient's nose, said holder comprising:
an upper nose base, a central bridge portion, and a lower tube securement tab;
said upper nose base having an adhesive backing for attachment to a patient's nose and said lower tube securement tab having an adhesive backing for attachment to the tube;
said central bridge portion positioned between said upper nose base and said lower tube securement tab;
said central bridge portion having a non-absorbent rear surface area including a structurally supportive non-adherent pad;
said pad functionally separating said upper nose base from said lower tube securement tab such that both said nose base and said lower tube securement tab can function independently of each other.
